(19) United States
(12) Patent Application Publication
(10) Pub. No.: US 2012/0065738 A1
(43) Pub. Date: Mar. 15, 2012

(54) Cortical Bone Spacers for Arthrodesis

(76) Inventor: Daniel Schulman, Phoenix, AZ (US)

(21) Appl. No.: 13/232,912

(22) Filed: Sep. 14, 2011

Related U.S. Application Data
(60) Provisional application No. 61/383,206, filed on Sep. 15, 2010.

(51) Int. Cl. A61F 2/30

(52) U.S. Cl. 623/23.44

(57) ABSTRACT

A spacer is used in a damaged bone to provide a contact surface that complements the shape of a cooperating bone so a corrective arthrodesis may be performed across the two bones. The spacer further may restore the length and, preferably, the shape of the damaged bone, particularly when a discrete bone segment has been removed. The spacer has a base having the length and shape of the removed bone segment, and a stem attached to the base for securing the spacer to the damaged bone. The spacer may have one or more fixation channels for receiving fixation devices, such as k-wires or bone pins. In a method of using the spacer, gradual osteotomies are performed on the damaged bone until bleeding bone is obtained. A stem hole is drilled to receive the stem, and the spacer is inserted and aligned with the damaged and cooperating bones.

### CROSS-REFERENCE TO RELATED APPLICATIONS

This application is a non-provisional and claims the benefit of U.S. Provisional Pat. App. Ser. No. 61/383,206, filed on Sep. 15, 2010.

### FIELD OF INVENTION

This invention relates to methods and devices for performing arthrodesis. This invention relates particularly to a surgical system that uses bone graft spacers in a corrective arthrodesis to address loss of bone stock.

### BACKGROUND

Arthrodesis is a surgical treatment that facilitates the fusion of two bones across a joint. It may be undertaken for many reasons, including relieving chronic joint pain, repairing damaged bone or surrounding structure, realigning the big toe after a bunionectomy, or correcting a failed orthopedic procedure such as an arthroplasty. It involves denuding both bones of surface cartilage where the bones will contact an implant, inserting the implant that serves as a bridge between the bones, and fixing the bones and implant together using a fixation device. Known implants include bone grafts, synthetic bone implants, and metal implants. The procedure induces joint ossification between the two bones.

Ossification may occur through contact healing or gap healing. In contact healing, two bony surfaces are brought into contact across the joint. This allows two stages of bone healing, bone union and bone remodeling, to occur simultaneously as new lamellar bone is formed in the axial direction to the rectus-aligned bones. In gap healing, the bony surfaces are separated by up to 6 mm of empty space. New bone forms across the gap by first developing in layers perpendicularly to the axial direction of the aligned bones until bone union across the gap is achieved. Later, the new bone undergoes remodeling, during which it is re-oriented along the axis of the aligned bones. Contact healing is generally understood to be more stable more quickly than gap healing, due to the direct formation of properly aligned new bone. It is therefore advantageous, particularly in weight-bearing joints such as those in the toes, to perform an arthrodesis that allows for contact healing.

Particularly in the cases of arthrodesis for joint repair and correction, it may be necessary to remove, by osteotomy, damaged or dead bone tissue at the site of the joint. For example, an arthroplastic implant may be rejected by the bone into which it is implanted, causing the surrounding bone tissue to necrotize. Similarly, an arthrodesis may fail, such as when force is applied to the surgical site before the bones have ossified. Some such failures leave dead or irreparable bone tissue at the site. In these cases, a corrective arthrodesis is needed. A corrective arthrodesis involves removing the dead or irreparable bone tissue caused by the failure of a previous arthrodesis, arthroplasty with implant, or arthroplasty without implant.

Unfortunately, the removal of dead bone tissue surrounding the joint in advance of a corrective arthrodesis may cause instability, deformity, or both, due to loss of bone length. These problems are exacerbated in the foot, where the bones are naturally short. Specifically, it is common that a centimeter or more of bone length must be removed in a hallux, and a half-centimeter or more in a lesser toe, which is a considerable percentage of the total length of metatarsal bones and phalanges. This loss of length can affect the weight-bearing capabilities of the toes, resulting in an unstable gait and other balance problems, and may further be uncomfortable or aesthetically displeasing to the patient. It would be advantageous to have a surgical system that can correct the shortcomings of corrective arthrodesis where significant lengths of bone must be removed. It would be further advantageous to employ the known advantages of bone autografts and allografts to further increase the stability of the arthrodesis through generation of new bone tissue where the joint was resected.

It is common that one of the two bones undergoing corrective arthrodesis is undamaged. In order to minimize the complications of osteotomy, it would be advantageous to denude the undamaged bone of cartilage but otherwise keep the bone intact. Unfortunately, the bones involved in arthrodesis are typically part of a ball-and-socket or condyloid joint, which means the ends of the bones are complementary: the end of one bone is concave and the end of the other, mated bone is convex. In contrast, typical metal implants and bone grafts are shaped like a block, wedge, or cylinder, but in any case have planar edges intended to contact planed bone on both ends. Such implants fail to provide an environment where new bone may be generated through contact healing unless the undamaged bone is also planed. It would be advantageous to match the end of the undamaged bone with a cooperating shape, as is the case in a normal joint, to maximize contact between the two bones without planing, through osteotomy, the undamaged bone.

Another disadvantage of known implants for arthrodesis is that the implant does not have a natural “bony” shape. Using a known implant, such as a block, wedge, or cylindrical implant, results in the healed toe having an unnatural appearance due to the visible absence of a knuckle. It would be advantageous to shape an arththrodesis device like the end of the bone it replaces so that the toe appears normal when healed.

Therefore, it is an object of this invention to provide an apparatus for corrective arthrodesis that limits the loss of bone length. It is a further object that the apparatus be composed of a material that is not harmful to the body. A further object is that the apparatus may be used on either or both sides of a joint. Another object is to provide an apparatus for arthrodesis that is bioabsorbable. A further object is that the apparatus provide for contact healing of the bones across the joint. A further object is that the apparatus can be used without removing any undamaged bone.

### SUMMARY OF THE INVENTION

A surgical system includes one or more spacers and methods of implanting each spacer to repair damaged living bone near a bone joint, where at least about 0.4 cm of bone length must be removed. Particularly, the spacers are adapted for restoring the original length of the bone, and preferably also the original shape. A spacer has a base and a stem. The base is shaped like the proximal or distal end of the bone to be repaired. Particularly, the base has a contact surface that is concave or convex, depending on the shape that the bone should have to complete the joint. The height of the base corresponds to the length of bone that was lost due to bone damage. The base may have pin apertures through which one or more pins may be inserted to secure the spacer to the bone. The stem is attached to or integral with the base and cooperates with a guide hole in the bone to align and attach the spacer to the bone. The base and stem may be cannulated to accommodate a K-wire or other fixation device. One or both of the base and stem may be fenestrated to increase the osteoconductivity of the spacer. Preferably, the spacer is made of human cortical bone, and most preferably is an allograft from a human cadaver.

In surgery, if one of the bones that form the joint is undamaged, cartilage is removed from the undamaged bone at the joint. Gradual osteotomies are performed on the damaged bone until bleeding bone is obtained. A guide hole is drilled into the damaged bone, and a sizing apparatus is inserted into the guide hole to obtain accurate measurements needed for the spacer's stem. The spacer may be modified to fit the guide hole. Depending on the procedure, a K-wire or other fixation device may be inserted into the spacer. The spacer is then inserted into the damaged bone using the guide hole. The spacer may be fixed in place using one or more K-wires, bone cement or another adhesive, or bone pins. The spacer may then be sculpted to match the damaged bone, the undamaged bone, or both, if necessary. The bones are properly aligned and then fixed into position with K-wire. The method may repair one or both bones in the joint.

### BRIEF DESCRIPTION OF THE DRAWINGS

FIG. 1 is a top front perspective view of the preferred spacer having a concave contact surface.

FIG. 2 is a top back perspective view of the spacer of FIG. 1.

FIG. 3 is a side view of the spacer of FIG. 1.

FIG. 4 is a top view of the spacer of FIG. 1.

FIG. 5 is a front view of the spacer of FIG. 1.

FIG. 6 is a rear view of the spacer of FIG. 1.

FIGS. 7A-7C are cross-sectional side views of a method of using the spacer of FIG. 1 for metatarsophalangeal arthrodesis of a human hallux.

FIG. 7D is a cross-sectional top view of the inserted spacer as shown in FIG. 7C.

FIG. 8 is a top front perspective view of a spacer having a convex contact surface.

FIG. 9 is a top back perspective view of the spacer of FIG. 8.

FIG. 10 is a side view of the spacer of FIG. 8.

FIG. 11 is a front view of the spacer of FIG. 8.

FIG. 12 is a rear view of the spacer of FIG. 8.

FIGS. 13A-13B are cross-sectional top views of a method of using the spacer of FIG. 8 for interphalangeal arthrodesis of a human lesser toe.

### DETAILED DESCRIPTION OF THE INVENTION

The present inventive surgical system includes one or more bone spacers for use in arthrodesis of bones across a joint, and methods of using the spacer to perform an arthrodesis. A spacer in the present system may be inserted in the proximal or distal end of either bone that forms the joint, as described herein, and is designed to replace a segment of the bone into which it is inserted, referred to herein as the “receiving bone.” The end of the receiving bone from which the bone segment is removed, which is also the end of the receiving bone that forms part of the joint, is referred to herein as the “damaged end.” The bone in the joint that cooperates with the receiving bone is referred to herein as the “cooperating bone.” The end of the cooperating bone that forms part of the joint is referred to as the “cooperating end.” It will be understood that the first or second bone, or both bones, of the joint may be receiving bones, and the proximal or distal end of either bone may be the damaged end or the cooperating end, as described below. The spacer restores the receiving bone substantially to its original length, and also preferably to its original shape, while providing a surface to contact the cooperating end of the cooperating bone for corrective arthrodesis.

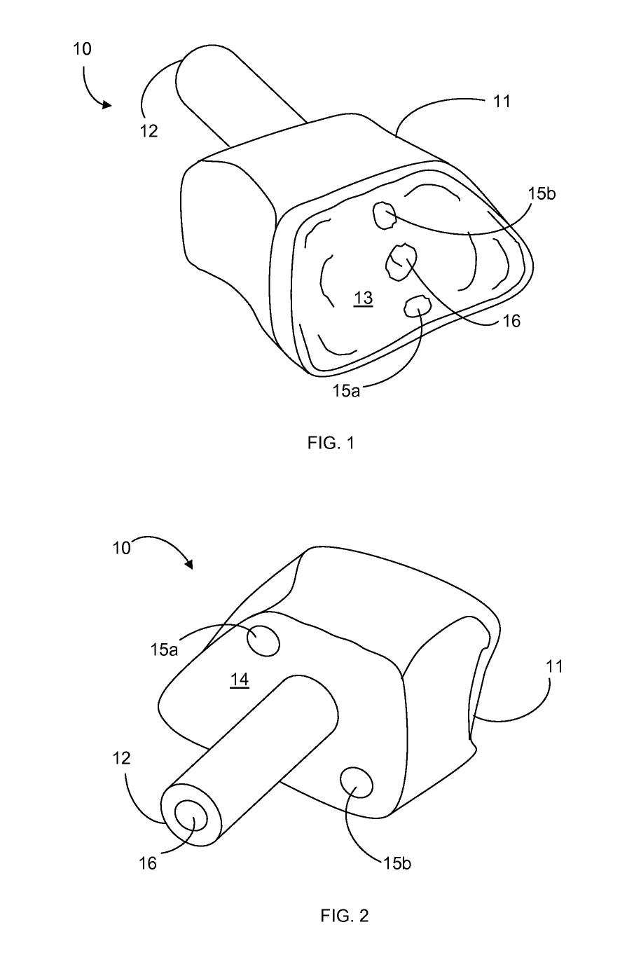

Referring to FIGS. 1-6, there is illustrated the preferred embodiment of a spacer 10 designed to be inserted into the proximal phalanx of a human hallux. The spacer 10 has a base 11 and a stem 12 that is attached to or integral with the base 11. Preferably, the stem 12 is integral with the base 11, the spacer 10 being molded, formed, or machined from a single piece of material. In a method of using the spacer 10, described in detail below, the stem 12 is inserted into the receiving bone so that the base 11 aligns with the receiving bone to replace the missing bone segment.

Generally, the shape of the base 11 is such that, when inserted into the receiving bone, the spacer 10 restores the receiving bone to the shape and size it would have had at the damaged end if no bone were removed. The base 11 has a contact surface 13 that contacts the cooperating end of the cooperating bone. The contact surface 13 is contoured to complement the contour of the cooperating end. Specifically, a “complementary” contact surface 13 is one that provides substantial contact with the cooperating end, with few, if any, gaps in contact. Thus, when the cooperating end has a convex contour, the contact surface 13 has a contour that is concave to substantially the same degree as the cooperating end is convex. For example, in the embodiment of FIGS. 1-6, the cooperating bone is the first metatarsal and the cooperating end is the distal end, or “head,” of the first metatarsal. The head of the first metatarsal is substantially convex and normally forms a ball-and-socket joint with the proximal end of the proximal phalanx, which is concave. The contact surface 13 is therefore substantially concave, to a degree that provides substantial, and preferably maximizes, surface-to-surface contact between the contact surface 13 and the convex surface of the first metatarsal head. The contact surface 13 preferably has a default concave shape that is chosen based on a typical degree of curvature of the first metatarsal head. However, the contact surface 13 may be modified, such as by using a burr-headed drill or cup reamer, to more precisely fit the metatarsal head of a specific patient.

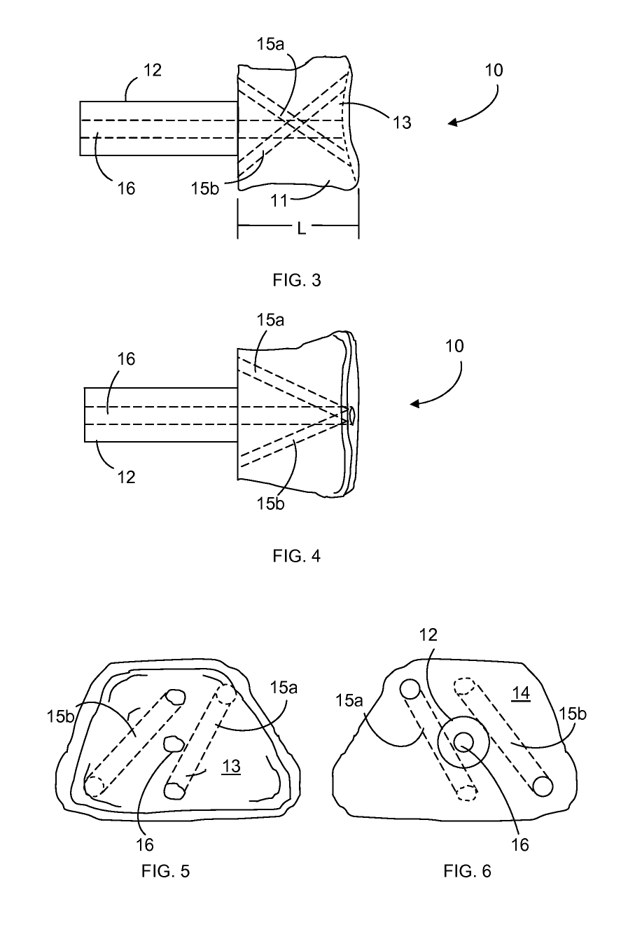

The base 11 further has a substantially planar abutting surface 14 that contacts the receiving bone when the spacer 10 is fully inserted. Preferably, the abutting surface 14 is angled relative to the stem 12 to maximize contact with a planed surface of the receiving bone as described below. For example, if the bone is planed perpendicular to its axis, the abutting surface 14 will be substantially perpendicular to the stem 12. In the preferred embodiment, the length L of the base 11 is substantially equal to the length of the bone segment that is removed. That is, the length L is determined so that inserting the spacer 10 into the receiving bone restores the receiving bone to the length that it would be if no bone had ever been removed. Generally, the spacer 10 replaces between about 0.4 and 2.0 cm, depending on the joint and bones involved, of bone length that would otherwise be lost during corrective arthrodesis if the spacer 10 were not used. The cross-sectional shape of the base 11 is substantially similar to that of the receiving bone. This shape may be uniform, but preferably is non-uniform throughout the length of the base 11, mimicking the variations in shape of the bone segment the spacer 10 is replacing. For example, the diameter of the proximal phalanx increases toward the proximal end to accommodate the socket shape of the metatarsophalangeal joint, and further develops its knobby shape, reflected in the appearance of the knuckle, for attachment of muscles. The spacer 10 is similarly shaped, as shown in FIGS. 3-6.

Alternatively, the spacer 10 may be shaped to correct an abnormality, such as bone misalignment. For example, in a corrective arthrodesis on a hallux that was previously subjected to a failed bunionectomy, the first metatarsal may still be deviated several degrees laterally from its normal alignment. Fixing a normally-shaped spacer 10 onto the bone would leave the toe abducted. Instead, the spacer 10 may be longer on the lateral side to compensate for the deviation and leave the phalanx substantially distally straight.

A portion or the entirety of the base 11 may be solid bone, such that an anchor may be securely inserted into the spacer 10 to retain sutures and secure one or more muscles or other soft tissue in place against the spacer 10. A portion or the entirety of the base 11 may be fenestrated to facilitate ingrowth of new bone from the surrounding living bone. To increase stability of the spacer 10, one or more fixation channels may be drilled through the base 11 and used to receive a fixation device, such as a k-wire or a bone pin. In the preferred proximal phalanx spacer 10 for the metatarsophalangeal joint, two supporting fixation channels 15a, 15b are drilled. The first supporting fixation channel 15a extends from the proximal-plantar-central aspect on the contact surface 13 to the distal-dorsal-medial aspect on the abutting surface 14. The second supporting fixation channel 15b extends from the proximal-dorsal-central aspect on the contact surface 13 to the distal-plantar-lateral aspect on the abutting surface 14. One or more fixation channels may be used, if desired, in configurations that accommodate the joint being resected and the stability desired.

The stem 12 extends out from the abutting surface 14 of the base 11. Preferably, the stem 12 is substantially cylindrical, but may alternatively be shaped like a prism having a polygonal base with three or more sides. The axis of the stem 12 is preferably substantially orthogonal to the abutting surface 14. Preferably, the stem 12 is positioned at or near the center of the abutting surface 14, so that the stem 12 extends into the central part of the receiving bone. The length of the stem 12 is sufficient to firmly secure the spacer 10 when it is inserted, and also to provide structural support as the surgical site is healing. The length of the stem may be up to 80% of the remaining length of the receiving bone. Preferably, the length of the stem 12 is at least 66% of the length of the remaining receiving bone.

The spacer 10 may be cannulated, creating a central fixation channel 16 that extends through the base 11 and spacer 12. The central fixation channel 16 provides seating for the primary fixation device. The central fixation channel 16 is preferably coaxial with the stem 12. Preferably, the central fixation channel 16 has a diameter that is substantially equal to or slightly larger than the diameter of the fixation device used. For the illustrated proximal phalangeal spacer 10, the preferred primary fixation device is a 0.062 inch diameter k-wire.

The spacer 10 is preferably made of an osteoconductive material. The material may also or alternatively be osteoinductive. Preferably, the spacer 10 is machined from a single piece of human cortical bone, which may be an autograft but is most preferably an allograft. Compared to existing metal, silicone, and polymer implants, the allografted cortical bone spacer exhibits superior integration with the surrounding living bone with a much lower chance of adverse tissue reaction or biofilm production. Another bioabsorbable material with similar properties to cortical bone, and further with osteoinductive properties, may be used in the alternative.

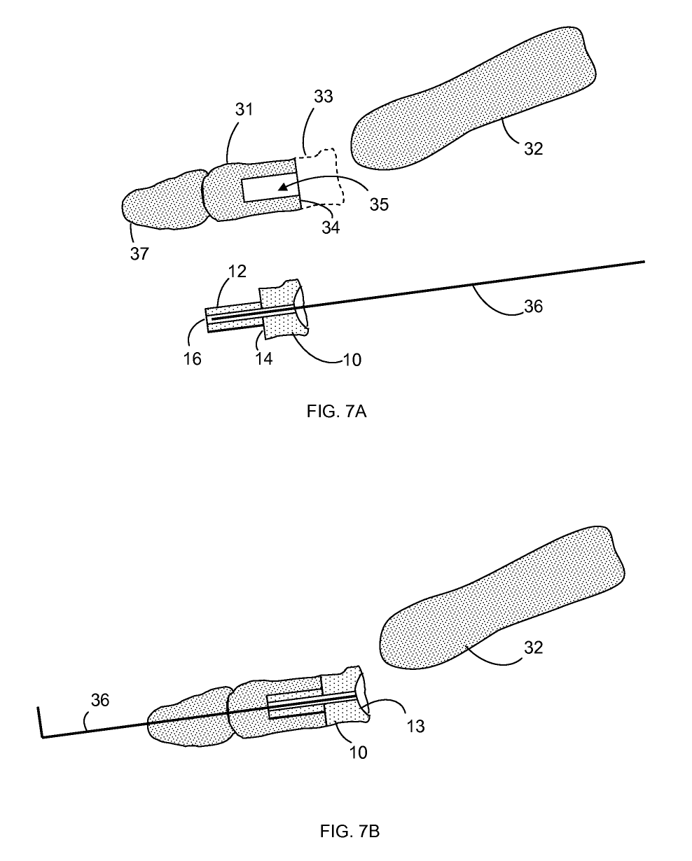

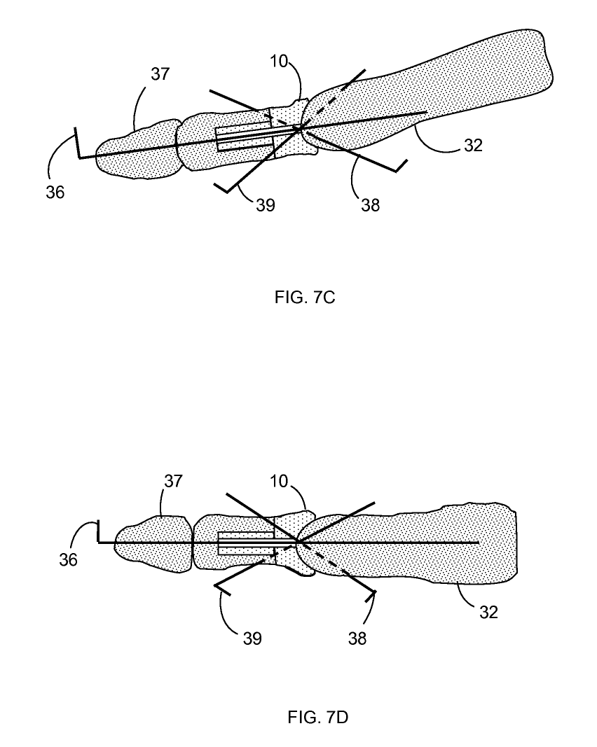

FIGS. 7A-D illustrate a method of using the spacer 10 of FIG. 1 for arthrodesis of a proximal phalanx 31, as the receiving bone, and a first metatarsal bone 32, as the cooperating bone, across the metatarsophalangeal joint. The head of the first metatarsal bone 32 is denuded of cartilaginous tissue, preferably by using an appropriately sized cone reamer, such as one sold by Ascension Orthopedics, Inc., a Texas corporation. If a previous implant was installed in the proximal phalanx 31, the previous implant is removed. The proximal phalanx 31 is prepared by making small osteotomies to the proximal end, preferably perpendicular to the longitudinal axis of the phalanx 31, until bleeding bone is seen. At least 1.0 cm, measured longitudinally, of the original bone will have been removed by the osteotomies combined with a previous surgery, if any. The osteotomies create a prepared surface 34 that will be substantially parallel to the abutting surface 14 of the spacer 10 when the spacer 10 is inserted. The dotted line of FIG. 7A indicates the bone segment 33 that is to be replaced.

A stem hole 35 is drilled distally into the shaft of the proximal phalanx 31, substantially along the longitudinal axis. The stem hole 35 is substantially equal in diameter to the stem 12, and is at least as deep as the stem 12 is long. In cases where a previous implant was removed, the shaft of the proximal phalanx 31 may contain toxins, necrotic tissue, or other damage. Preliminary repair may be needed, wherein the damaged tissue is removed to form a bone void, and the bone void is filled with bone putty as is known in the art. Once the bone putty has set, the stem hole 35 may be drilled.

Once the proximal phalanx 31 is prepared, a sizing device is used to determine the exact length L for the base 11 of the spacer 10. Preferably, the sizing device is a sterilized test spacer made of plastic or metal. The test spacer may be one of a set of test spacers being similarly shaped to the spacer 10 and having different base lengths. The preferred set of test spacers have base lengths increasing in increments of 2-4 mm from 1.0 cm to 2.0 cm. The test spacer may have a stem to insert into the stem hole during use, or the test spacer may be held in place by hand or using a clamping tool. A radiograph of the original joint, if available, may be used to assist in determining the correct length L for the base 11. The appropriate spacer 10 is selected and loaded with the primary fixation device. Preferably, loading the spacer 10 is done by inserting a 0.062 inch primary k-wire 36 into the central fixation channel 16 from the proximal end of the spacer 10, such that the primary k-wire 36 is seated in the central fixation channel 16 without protruding from the stem 12. The stem 12 is then inserted into the stem hole 35 and the spacer 10 is driven into place, until the abutting surface 14 contacts the prepared surface 34. The spacer 10 may be driven into place by hand or with light compression using a convex-faced tool that applies pressure evenly to the contact surface 13. The appropriate alignment is then verified.

With the spacer 10 in place, the primary k-wire 36 is driven distally through the proximal and distal phalanges 31, 37 and retrograded to the contact surface 13. The contact surface 13 is then brought into contact with the denuded head of the first metatarsal 32 and the bones 31, 32 are aligned according to appropriate dorsiflexion and abduction positioning across the resected joint. Once the bones 31, 32 are aligned, the primary k-wire 36 is driven proximally through the head and into the shaft of the first metatarsal 32. The primary k-wire 36 is then cut and secured at an appropriate length.

If additional stabilization of the fusion site is desired, the supporting fixation channels 15a, 15b may receive supporting fixation devices, such one or more supporting k-wires 38, 39. The preferred supporting k-wires are 0.045 inches in diameter. The supporting k-wires 38, 39 may be inserted before or after aligning the bones and driving the primary k-wire 36 into the metatarsal head. Additionally or alternatively, the supporting fixation devices may include one or more bone pins. The preferred bone pins are 2.0 mm in diameter and are made of cortical bone. Where bone pins are used, a cannulated drill creates guide holes over the supporting k-wires 38,39 and the bone pins are inserted into the guide holes after the supporting k-wires 38,39 are removed. Additionally or alternatively, the supporting fixation devices may include one or more anchors configured to hold one or more suture lines away from the fusion site. The crossing configuration of the supporting fixation devices with the primary fixation device at the point of fusion aids in stabilization. Where anchors are used, sutures may be connected to muscle or other soft tissue through the anchor, tying the soft tissue and securing it in place across the point of fusion to provide additional musculoskeletal support for bones drawn into contact.

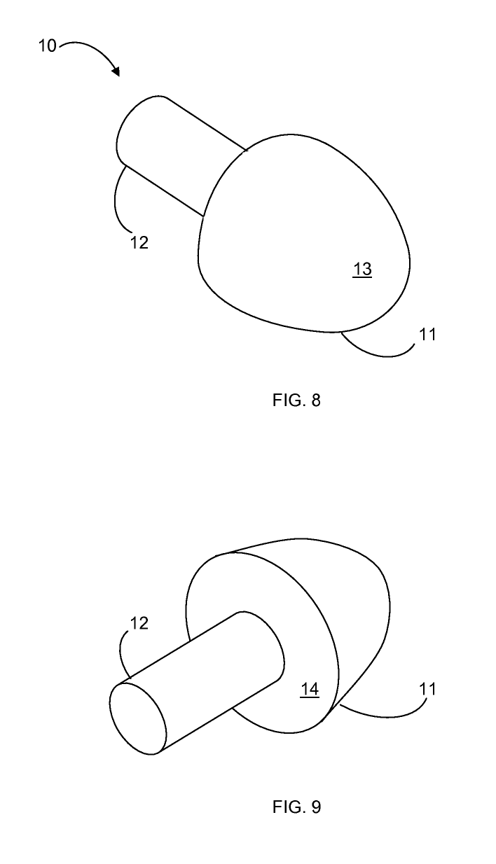

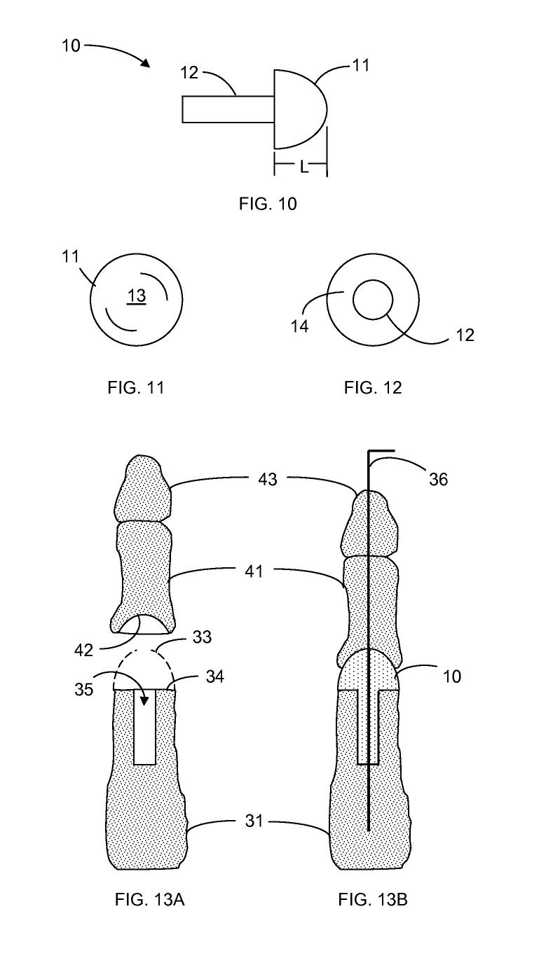

Due to the size of the bones involved in arthrodesis of certain joints, for example the proximal interphalangeal joint, drilling or cannulating the spacer 10 may weaken its structure more than aid it. Therefore, in other embodiments, the spacer 10 may have more or fewer fixation channels, and further may or may not be cannulated. For example, FIGS. 8-12 illustrate a spacer 10 that is inserted into the distal end of a proximal phalanx for arthrodesis of the proximal interphalangeal joint. The base 11 has a substantially circular profile. See FIG. 10. The contact surface 13 is convex to cooperate with the proximal end of the middle phalanx, which is concave. One or both of the base 11 and stem 12 may be fenestrated to encourage new bone ingrowth into the spacer 10.

FIGS. 13A-B illustrate arthrodesis of the proximal interphalangeal joint using the spacer 10 of FIGS. 8-12. The concave surface 42 on the proximal end of the middle phalanx 41 is denuded of cartilaginous tissue using a power burr or a cup reamer. The prepared surface 34 and stem hole 35 are prepared as described above, and a sizing tool is used to determine the proper size of the base 11. The spacer 10 is then inserted into the proximal phalanx 31 in rectus alignment.

The primary fixation device, specifically the primary k-wire 36, is inserted after the spacer 10 is inserted. Preferably, a drill is used to drive the primary k-wire 36 through the base 11 and stem 12 and into the receiving bone. In this example, only the primary k-wire 36 is used, although in still other embodiments the supporting fixation devices may be used. The primary k-wire 36 is driven distally through the middle phalanx 41 and lesser distal phalanx 43 until retrograded to the concave surface 42. The concave surface 42 is then brought into contact with the contact surface 13 and the bones 31, 41 are placed in rectus alignment. The primary k-wire 36 is then driven proximally through the spacer 10, substantially coaxially with the stem 12, and into the shaft of the proximal phalanx 31. The primary k-wire 36 is then cut to the desired length and secured.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims

What is claimed is:

1. A device for insertion into a first bone in a corrective arthrodesis of a joint between the first bone and a second bone, the device comprising:
a. a base having a length substantially equal to the length of a bone segment removed from a damaged end of the first bone, the base comprising a contact surface configured to contact the second bone and complement the shape of the second bone where the contact surface contacts the second bone; and
b. a stem attached to the base.

2. The device of claim 1 wherein the base further comprises an abutting surface that contacts the first bone when the device is inserted into the first bone.

3. The device of claim 2 wherein the abutting surface is planar.

4. The device of claim 1 wherein the stem and base are made of cortical bone.

5. The device of claim 1 wherein the first bone is a proximal phalanx, the second bone is a middle phalanx, and the contact surface is convex and cooperates with a concave portion of the second bone.

6. The device of claim 1 further comprising one or more fixation channels.

7. The device of claim 6 wherein one of the fixation channels is a central fixation channel passing through the stem and the base and being coaxial with the stem.

8. The device of claim 6 wherein one or more of the fixation channels is a supporting fixation channel.

9. The device of claim 7 wherein the first bone is a proximal phalanx, the second bone is a metatarsal bone, and the contact surface is concave and cooperates with a convex portion of the second bone.

10. The device of claim 1 wherein the base is shaped substantially similarly to the bone segment such that the first bone is substantially restored to its original size and shape when the device is inserted into the first bone.

11. The device of claim 10 wherein:
a. the base is integral with the stem;
b. the base further comprises a planar abutting surface that contacts the first bone when the device is inserted into the first bone; and
c. the stem is substantially cylindrical and projects from the abutting surface.

12. The device of claim 11 wherein the base is between 0.4 cm and 2 cm long.

13. The device of claim 12 wherein the base and stem are made of cortical bone.

14. The device of claim 13 further comprising a central fixation channel passing through the stem and the base and being coaxial with the stem.

15. The device of claim 14 further comprising one or more supporting fixation channels.

16. A method for performing a corrective arthrodesis of a first bone and a second bone across a joint, the method comprising:
a. inserting a spacer into the first bone to restore the first bone to its undamaged length, the spacer comprising:
i. a base having a length substantially equal to the length of a bone segment removed from a damaged end of the first bone, the base comprising a contact surface configured to contact the second bone and complement the shape of the second bone where the contact surface contacts the second bone; and
ii. a stem attached to the base; and
b. positioning the spacer in contact against the second bone so that the first bone and second bone may fuse across the joint.

17. The method of claim 16 further comprising:
a. performing gradual planar osteotomies on the first bone from the damaged end until bleeding bone is obtained; and
b. drilling a stem hole into the first bone from the damaged end;
wherein inserting the spacer into the first bone comprises inserting the stem into the stem hole.

18. The method of claim 16 wherein the base is shaped substantially similarly to the bone segment, the method further comprising aligning the base with the first bone so that the base restores the first bone to its undamaged length and shape.

19. The method of claim 16 further comprising:
a. driving a primary fixation device through the first bone and the spacer;
b. bringing the contact surface into contact with the denuded cooperating end; and
c. driving the primary fixation device through a portion of the second bone so that the primary fixation device fixes the first bone, spacer, and second bone in place.

20. A method for performing a corrective arthrodesis across a joint between a damaged end of a first bone and a cooperating end of a second bone, the method comprising:
a. preparing the first bone to receive a spacer, the spacer being made of cortical bone and comprising:
i. a base having a length substantially equal to the length of a bone segment removed from a damaged end of the first bone, the base comprising a contact surface configured to contact the second bone and complement the shape of the second bone at the point of contact; and
ii. a cylindrical stem integral with the base and projecting orthogonally from an abutting surface of the base;
and the preparing comprising:
iii. performing gradual planar osteotomies on the first bone from the damaged end until bleeding bone is obtained; and
iv. drilling a stem hole into the first bone from the damaged end, the stem hole having a diameter substantially equal to the diameter of the stem;
b. denuding the cooperating end of the second bone of cartilage and tissue;
c. inserting the stem of the spacer into the stem hole until the abutting surface contacts the first bone;
d. aligning the base with the first bone so that the base restores the first bone to its undamaged shape;
e. driving a primary fixation device through the first bone and the spacer;
f. bringing the contact surface into contact with the denuded cooperating end; and
g. driving the primary fixation device through a portion of the second bone so that the primary fixation device fixes the first bone, spacer, and second bone in place.
